(19) United States
(12) Patent Application Publication
(10) Pub. No.: US 2005/0018138 A1
(43) Pub. Date: Jan. 27, 2005

(54) Advanced astigmatism testing method

(76) Inventor: Robert Grainger, Florissant, MO (US)

Correspondence Address: Robert J. Grainger, 3765 Santiago Drive, Florissant, MO, 63033, US

(21) Appl. No.: 10/414,626

(22) Filed: Jul. 23, 2003

(51) Int. Cl. A61B 3/02

(52) U.S. Cl. 351/222

(57) ABSTRACT

The Advanced Astigmatism Testing Method provides a great improvement over standard Snellen charts in testing vision patients with astigmatism. An ophthalmologist/optometrist has the person view an illuminated sheet of white paper with a pair of L's and placed on the viewing wall. A person with astigmatism will see secondary images of the L's offset horizontally and/or vertically. The vision specialist will then vary lens settings as the patient reports the movement of the vertical or horizontal offset. It has been validated that after the vertical offset diminishes completely that the horizontal offset can likewise be reduced to zero. The lens settings are recorded and serve as the basis for the lens prescription.

### CROSS REFERENCE TO RELATED APPLICATION

This non-provisional utility patent application claims the priority benefit of provisional application: 60/373,499, filing date: Apr, 17, 2002 applicant: Robert John Grainger (USA citizen), residence: 3765 Santiago Drive, Florissant, Mo. 63033.

### STATEMENT REGARDING FEDERALLY SPONSERED RESEARCH OR DEVELOPMENT

Not applicable.

### BACKGROUND OF THE INVENTION

### FIELD OF THE INVENTION: OPTOMETRY

This invention deals with a problem encountered by people with astigmatism (double vision) during vision exams. It is very difficult to distinguish the small letters in a row when each has an extra image either separated or slightly offset. Determining the difference in lens setting choices becomes particularly difficult if the offsets are in two directions.

### BRIEF SUMMARY OF THE INVENTION

In this new method the basic object to be viewed by the astigmatic person sitting in the optometrist's chair consists of two bars arranged in an L shape (FIG. 1A). The L could appear on an 8½×11 sheet of paper on the normal viewing wall. A view of typical “wide” and “narrow” L-shapes is shown in FIGS. 1A and 1B. The dimension of the L-shape is not critical. A typical size of the wide bars would be 2 inches by 3/8 inch. The narrow L should have a width one-fourth that of the wide L for both horizontal and vertical elements.

For a person with astigmatism the images of the vertical and horizontal portions of the L will be perceived differently. The fainter secondary images will ordinarily shift by unequal amounts for the vertical and horizontal bars.

Both coarse and fine testing can be performed using the above-mentioned procedure. Coarse testing would be performed initially using broad bars. Fine testing would use narrow bars for more precise lens settings.

### DETAILED DESCRIPTION OF THE INVENTION

A computer reconstruction of an example of the primary and secondary images of the wide and narrow L's as viewed by an astigmatic patient is presented in FIG. 2.

Testing is accomplished by an ophthalmologist/optometrist simply by placing an 8½×11 sheet of paper with the two L's on the wall of his examining room and illuminated with a spotlight. The patient in the chair then declares which of the first two lens settings improves the image. Continuing this process will bring the primary and secondary image of each L closer together until they overlap completely for either the horizontal or vertical offset. The process is then repeated for the other offset component. A striking feature of the final images is that the horizontal and vertical portions of an L, which had been gray, end up as completely black and quite pronounced. The recorded lens settings can then be used for a vision prescription.

### LISTING OF FIGURES

FIG. 1A: Wide L-Shape

FIG. 1B: Narrow L-Shape

FIG. 2: Example of wide and narrow L's as viewed by astigmatic patient

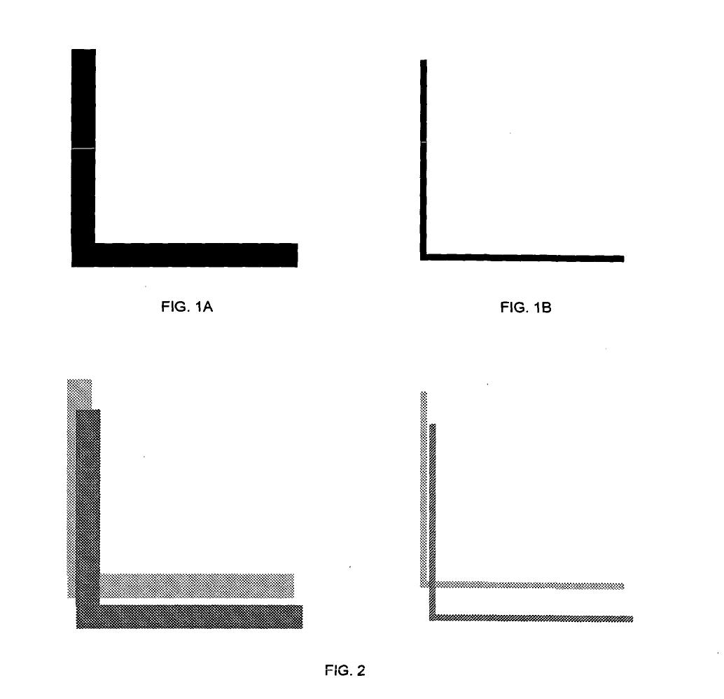

1. I claim that I was able to validate that the horizontal and vertical astigmatism offsets can be reduced to zero as the result of the interaction of the patient's observations of L-shapes and the ophthalmologist's lens selections during performance of the Advanced Astigmatism Testing Method. As stated in a different way the Advanced Astigmatic Testing Method can allow an astigmatic patient view of L's to change from, for example, FIG. 2 to FIG. 1 during the vision exam.
